(19) United States
(12) Patent Application Publication
(10) Pub. No.: US 2015/0246154 A1
(43) Pub. Date: Sep. 3, 2015

(54) PROCESS AND DRESSING

(71) Applicant: Christian Stephenson, Wilmslow (GB)

(72) Inventor: Christian Stephenson, Wilmslow (GB)

(21) Appl. No.: 13/979,165

(22) PCT Filed: Jan. 11, 2012
(86) PCT No.: PCT/EP12/50379

§ 371 Date: Jul. 8, 2014

(30) Foreign Application Priority Data
Jan. 11, 2011 (GB) 1100425.6

(51) Int. Cl. A61L 15/28; A61F 13/00; C08B 37/08

(52) U.S. Cl. CPC A61L 15/28; C08B 37/003; A61F 13/00991; A61F 2013/00285

(57) ABSTRACT

An anti-microbial absorbent chitosan, optionally in the form or fibres, derivatised by reaction with an optionally substituted alkylene oxide, a non-woven fabric and an absorbent device comprising the derivatised chitosan, methods for the preparation of such a chitosan, non-woven fabric and an absorbent device, and the use of said absorbent device in wound care.

The present invention relates to an anti-microbial component of a absorbent device, to a method for the preparation of such an component, and in particular to a method for the preparation of a novel hydrophilic, anti-microbial derivatised chitosan, including in the form or fibres, and a method for applying metal ions to the chitosan, and also to an absorbent device comprising the component, a method for the preparation of such an absorbent device, and the use of said absorbent device.

The term ‘absorbent medical device’ as used herein includes wound dressings for acute wounds, including surgical wounds, and chronic and burn wounds, ostomy devices, surgical and dental sponges, and absorbent pads for the personal care sector, particularly for disposable sanitary devices such as nappies (diapers), disposable nappies and training pants, feminine care products, for example, tampons, sanitary towels, or napkins and pant liners, and incontinence products. It in particular includes (but is not limited to) an absorbent medical device comprising as an absorbent material, a water-insoluble organic or inorganic acyl substituted hydrocarbyl chitosan.

Reference to a “derivatised chitosan” herein is to a substance which is a chitosan which is substituted by salified hydroxy-(organic or inorganic acyl)-hydrocarbyloxy groups in place of at least a proportion of the chitosan hydroxyl functions.

Reference to a “further derivatised chitosan” herein is to a substance which is a chitosan which is substituted by basified hydroxyethyl esters of (organic or inorganic acyl)-hydrocarbyloxy groups in place of at least at least a proportion of the chitosan hydroxyl functions. The ethyl moiety is optionally substituted by hydrocarbyl groups.

The term ‘organic or inorganic acyl’ as used herein includes organic acid residues such as carbonyl and inorganic acid residues such as sulphonyl. It will be appreciated that where such residues are linked by a single bond to an O atom they form an acylate residue of an organic or inorganic oxoacid.

By “substantially water-insoluble” herein is meant that when a material, for example a further derivatised chitosan is exposed to an excess of an aqueous medium it does not dissolve into solution, or at least that dissolution is so low as to have no significant effect on the physical properties of the polymer.

Reference to the “absorbency” of a further derivatised chitosan or a product such as a non-woven fabric consisting essentially of the fibres herein is to the capacity of the derivatised chitosan or product to take up fluid.

In the case of the present further derivatised chitosan fibres, fluid is absorbed into the internal fibre structure and the fibre swells. In the case of a non-woven fabric consisting essentially of the fibres, the overall absorptive capacity is sensitive to the sizes and interconnectivity of the inter-fibre volumes within the fabric, and hence its process for manufacture. However, in the case of the present fibres and non-woven fabrics, the absorbencies will not differ greatly.

Measurement of the overall absorptive capacity of an absorbent material or of a absorbent device, in particular a medical absorbent device, comprising such material is a convenient and effective process for determining the effectiveness of the absorbent material or device for absorbent applications, such as wound dressings.

Wound-contacting materials in dressings should preferably have good absorbency whilst being substantially insoluble, should maintain their integrity when wet, not adhere to the wound, and have antimicrobial properties.

Polyacetylglucosamine, also known as chitin, is an amylose widely existing in nature. It is a major composition of fungal cell wall and carapace of shrimps, crabs and insects. It may be at least partially deacetylated to produce free hydroxyl functions, and so to form chitosans with varying degrees of deacetylation and free hydroxyl functions. It will be appreciated that the composition of any chitosan may vary with the source, the degree of deacetylation and the molecular weight of the chitin starting material that is used in its production. There are 2 acetylated hydroxyl functions per glucosamine unit of chitin. Any deacetylated hydroxyl function may be substituted randomly throughout the corresponding chitosan.

Substitution may take place at either hydroxyl position in the glucosamine units of the chitosan macromolecule, in any distribution up to the maximum degree of substitution that is possible.

There is also one amine function per glucosamine unit of chitin and any chitosan. It is believed that in the present products and processes, the amine functions may be substituted randomly throughout a relevant chitosan in any distribution up to the maximum degree of substitution that is possible.

Thus, when used herein with respect to a derivatised chitosan, average degree of substitution refers to the mean number of hydroxyl groups in any positions that are substituted, that is, the mean number of moles of substituents per mole of D-glucosamine unit in the chitosan polymer. The maximum degree of substitution in such a chitosan derived from a chitin which has been 100% deacetylated is therefore 2, when each D-glucosamine unit is substituted at both hydroxyl positions.

When used herein with respect to a further derivatised chitosan, average degree of substitution refers to the mean number of hydroxyl and amine groups in any positions that are substituted, that is, the mean number of moles of substituents per mole of D-glucosamine unit in the chitosan polymer. The maximum degree of substitution in such a chitosan derived from a chitin which has been 100% deacetylated is therefore 3, when each D-glucosamine unit is substituted at both hydroxyl positions and the amine position.

It is known to produce and process a partially carboxymethylated modified chitosan fibre to an absorbent non-woven fabric component of a wound dressing by fibre opening, web formation and needling the fibre finish on the surface of the chitosan. It is also known to sterilise a dry material of this type with ethylene oxide.

A first technical problem of the prior art is that the high aqueous absorbency of the partially carboxymethylated chitosan fibre causes difficulty in fibre opening and web formation during subsequent processing of partially carboxymethylated chitosan amine salt fibre to a non-woven fabric by a non-woven technique.

A second technical problem of the prior art is that after being processed by a non-woven processing machine, the fibre finish on the surface of the partially carboxymethylated chitosan fibre makes the non-woven fabric hydrophobic. The hydrophobicity adversely affects the fluid absorbency of the dressings of the prior art, in particular from acute wounds, including surgical wounds, and chronic and burn wounds.

To be useful in an absorbent device, the fibres of an absorbent material may suitably have an absorbency of 8 to 30 grams per gram (g/g) standard solution, preferably 12 to 27 g/g, and more preferably 16 to 23 g/g.

It is therefore an object of the present invention to provide a therapeutically active derivatised chitosan fibre which is readily processable in fibre opening and web formation during subsequent processing to a non-woven fabric component of such a wound dressing, thus overcoming the corresponding disadvantage of the prior art materials.

It is also an object of the present invention to overcome the disadvantage of the hydrophobicity of prior art fabrics, and to provide a therapeutically active derivatised chitosan fibre for a dressing component which has a wound-facing surface which is not hydrophobic and has good absorbency of fluids, in particular from acute wounds, including surgical wounds, and chronic and burn wounds, and which also meets the above criteria for good absorbency of fluids, in absorbent devices, and in particular in advanced wound dressings.

Surprisingly, we have now found that the processability of a derivatised chitosan may be improved, and in particular the absorbency of the derivatised chitosan and of an absorbent device comprising it may be significantly improved by treatment with an optionally substituted oxirane, preferably in the presence of water or water vapour.

Thus, in order to solve the above technical problems, according to a first aspect of the present invention, there is provided a further derivatised chitosan wherein in place of at least part of the chitosan hydroxyl groups the chitosan is substituted by basified hydroxyethyl ester groups of formula (I)

HO—(R¹)(R²)C—C(R³)(R⁴)—O*—R—O— (I)

wherein each of R¹, R², R³ and R⁴ is H or an optionally substituted hydrocarbyl group, and

R is an organic or inorganic acyl hydrocarbyl residue, orientated such that the basified HO—(R¹)(R²)C—C(R³)(R⁴)— moiety forms an ester linkage with the oxygen atom marked *.

O*—R may suitably be a hydrocarbyl acylate residue of an organic or inorganic oxoacid, for example an alkane acylate of an organic or inorganic oxoacid.

O*—R may thus suitably be, for example, an alkanoate residue, preferably a lower alkanoate residue with 2 to 6 carbon atoms, such as an acetate, glyoxylate, propionate, pyruvate or butyrate, preferably acetate, propionate or butyrate preferably attached in the 2-position to the chitosan oxy group.

The alkanoate moiety may be branched or unbranched, and hence suitable butyrates may be n-butyrate or iso-butyrate.

The alkanoate residue that is most preferred is an acetate residue. The corresponding ester group of formula (I) may also be referred to as a carboxymethyl ester.

O*—R may also suitably be, for example an alkanesulphonate residue, preferably a lower alkanesulphonate with 2 to 6 carbon atoms, such as a methanesulphonate, ethanesulphonate, or propanesulphonate, preferably attached in the 1-position to the chitosan oxy group. and more preferably methanesulphonate. The alkane moiety may be branched or unbranched, and hence suitable propane sulphonates may be propane-1- or 2-sulphonate, and butanesulphonates may be butane-1-sulphonate, 2,2-dimethylethane-1-sulphonate or 1,2-dimethylethane-1-sulphonate. The alkane sulphonate substituent group that is most preferred is methane sulphonate. The corresponding ester group of formula (I) may also be referred to as a sulphonatomethyl ester.

O*—R may also suitably be an arenecarboxylate residue, such as a benzoate or toluate, substituted by a nucleophilic leaving group T.

O*—R may also suitably be an arenesulphonate residue, such as benzenesulphonate or toluenesulphonate, substituted by a nucleophilic leaving group T.

The basifying moiety for the ester hydroxyl function is preferably a Group IA cation, preferably a sodium cation (as hereinafter defined).

Each of R¹, R², R³ and R⁴ is H or a hydrocarbyl group, preferably H.

Each of R¹, R², R³ and R⁴ independently may be an alkyl group, preferably an optionally substituted lower alkyl group with 1 to 6 carbon atoms, such as a methyl, ethyl or propyl, preferably methyl.

The alkyl moiety may be branched or unbranched, and hence suitable propyl may be n-propyl or iso-propyl.

Each of R¹, R², R³ and R⁴ independently also suitably be, for example an acyl group, preferably a lower acyl group with 1 to 6 carbon atoms, such as a formyl or acetyl group. All of R¹, R², R³ and R⁴ together may have 1 to 6 carbon atoms, preferably 1.

Each of R¹, R², R³ and R⁴ independently may also suitably be an aryl group, such as phenyl or tolyl, preferably phenyl. Preferably only one of R¹, R², R³ and R⁴ is phenyl.

Most preferably each of R¹, R², R³ and R⁴ is H.

Depending on how the further derivatised chitosan of the invention is produced other functions may be substituted. For example, when produced by the second (epoxidation) process of the second aspect of the present invention, described further hereinafter, the chitosan amine groups are converted at least in part to groups of formula (IIa):

HO—(R¹)(R²)C—C(R³)(R⁴)—NH— (IIa)

so that the further derivatised chitosan product (as defined hereinbefore) is believed to be one in which in the D-glucosamine units of the derivatised chitosan
a) in place of at least part of the chitosan hydroxyl groups the chitosan is substituted by basified hydroxyethyl ester groups of formula (I)

HO—(R¹)(R²)C—C(R³)(R⁴)−O*—R—O— (I); and
b) the amine groups are substituted at least in part by optionally basified groups of formula (IIa):

HO—(R¹)(R²)C—C(R³)(R⁴)— (IIa)

The basifying moiety is preferably a Group IA metal cation, in particular a sodium cation.

It will be appreciated that if such a further derivatised chitosan is contacted with an aqueous medium, exchange of basifying groups between basified and non-basified HO— groups may occur, and that the definitions of the groups of formulae (I) and (IIa) should be interpreted accordingly.

Also for example, when prepared by the first process of the second aspect of the present invention as described further hereinafter, the further derivatised chitosan product (as defined hereinbefore) is believed to be one in which on the D-glucosamine units of the derivatised chitosan
a) in place of at least part of the chitosan hydroxyl groups the chitosan is substituted by basified hydroxyethyl ester groups of formula (I)

HO—(R¹)(R²)C—C(R³)(R⁴)—O*—R—O— (I); and
b) in place of at least part of the other chitosan hydroxyl groups the chitosan is substituted by basified groups of formula (II):

HO—(R¹)(R²)C—C(R³)(R⁴)—O— (II); and.
c) the amine groups are substituted at least in part by optionally basified groups of formula (IIa):

HO—(R¹)(R²)C—C(R³)(R⁴)— (IIa)

Again, the basifying moiety is preferably a Group IA metal cation, in particular a sodium cation.

It will be appreciated that if such a further derivatised chitosan is contacted with an aqueous medium, exchange of basifying groups between basified and non-basified HO— groups may occur, so that for example they may transfer from any moiety of formula (I) or (II) to that of formula (IIa), and that the definitions of the groups of formulae (I), (II) and (IIa) should be interpreted accordingly.

The further derivatised chitosan is substantially water-insoluble and preferably in the form of fibres. Fluid is absorbed into the internal fibre structure and the fibre swells. The further derivatised chitosan fibres of the invention are highly advantageous for use as absorbent materials in absorbent devices because they are non-toxic, odourless and compatible with human tissue and do not cause any immune response. They also have anti-microbial, antiphlogistic, haemostatic and antalgic capability, and facilitate wound healing.

The therapeutically active, absorbent further derivatised chitosan fibres of the present invention meet the criteria for good absorbency of fluids in absorbent devices, and in particular in advanced wound dressings. The derivatised chitosan fibres may suitably have an absorbency of 8 to 30 grams per gram (g/g) of saline solution, often 12 to 27 g/g, and more often 16 to 23 g/g.

It will be appreciated that the maximum possible degree of the above substitution will depend on the proportion of free deacetylated hydroxyl groups in the chitosan available for substitution, and on the other substituent groups present. The degree of deacetylation is often from 60 to 100%. Means of achieving a suitable degree of substitution for the further derivatised chitosan to be substantially water-insoluble are described in connection with the second process of the second aspect of the invention.

In a second aspect, the present invention provides a first process for producing a further derivatised chitosan comprising alkalised hydroxyethyl esters of (organic or inorganic acyl)-hydrocarbyloxy groups, in which the ethyl moiety is optionally substituted by hydrocarbyl groups, which comprises contacting chitosan
(a) with a base in an aqueous or non-aqueous medium, and
(b) with a solution in an aqueous or non-aqueous medium of a salt of the formula (III):

M-O—R-T (III)
wherein
M is a Group IA metal cation;
O—R is a hydrocarbyl oxoacid anion residue; and
T is a nucleophilic leaving group; and
(c) isolating and washing the product of steps (a) and (b), and
(d) reacting the product of step (c) with an alkylene oxide of the formula (IV):

R¹(R²)V(R³)R⁴ (IV)
wherein
V is an ethylene oxide moiety, and
each of R¹, R², R³ and R⁴, is H or an optionally substituted hydrocarbyl group, and R¹ and R² are at the 2-positions, and R³ and R⁴ are at the 3-positions, on V.

The term ‘derivatised chitosan’ as used in this context refers to the product of reaction steps (a) to (c) above, and the term ‘further derivatised chitosan’ as used herein refers to the product of reaction step (d).

As noted hereinafter, the further derivatised chitosan of the invention is often in the form of fibre tow. It is therefore preferred that the chitosan starting material of this invention is in a form corresponding to that of the desired further derivatised chitosan, in particular as fibres.

The derivatised chitosan fibre product from steps (a) to (c) of this process is one in which one or both of the hydroxyl groups on the D-glucosamine units of the chitosan are converted at least in part to salified O—R—O— groups (etherification). The base is preferably an alkali metal hydroxide, in which case the metal cation is preferably the same as M in the compound of formula (III), preferably an alkali metal hydroxide, such as sodium hydroxide, and these hydroxyl functions are preferably converted to M-O—R—O— groups. The other hydroxyl groups on the D-glucosamine units of the chitosan are generally converted to hydroxyl group derivatised by the base (alkalisation), preferably M-O— groups.

Either substitution may take place at either hydroxyl position in the chitosan macromolecule, in any distribution up to the maximum degree of substitution that is possible.

The relevant derivatised hydroxyl groups in all positions are described hereinafter as M-O—R—O— groups and M-O— groups on the D-glucosamine units in the derivatised chitosan polymer.

However, the term CM-O—R—O— groups' as used herein includes hydroxyl groups on the D-glucosamine units of the chitosan that have been converted to salified O— R—O— groups (etherification), being only preferably M-O—R—O— groups in which the metal cation is preferably the same as M in the compound of formula (III).

The term ‘M-O— groups’ as used herein includes hydroxyl groups on the D-glucosamine units of the chitosan that have been converted to hydroxyl group derivatised by a base (alkalisation), being only preferably M-O— groups.

The average degree of substitution here refers to the mean number of hydroxyl groups in all positions converted to M-O—R—O— groups, that is, the mean number of moles of M-O—R—O— groups per mole of D-glucosamine unit in the chitosan polymer. As noted hereinbefore, the maximum possible degree of the above substitution will depend on the proportion of free deacetylated hydroxyl groups in the chitosan available for substitution, and on the other substituent groups present. The degree of deacetylation is often from 60 to 100%.

The average degree of substitution by all such groups is suitably less than 0.8, preferably less than 0.7, more preferably less than 0.6 for the derivatised chitosan to be substantially water-insoluble. The average degree of substitution in the further derivatised chitosan polymer of the present invention may more suitably be from about 0.1 to about 0.4, for example from about 0.15 to about 0.35, such as from about 0.2 to 0.3. The average molecular weight of the chitosan is often between 3800 to 20,000 daltons.

In the derivatised chitosan intermediate:

M may suitably be a sodium or potassium cation, preferably a sodium cation.

O—R may suitably be a hydrocarbyl acylate of an organic or inorganic oxoacid, for example an alkane acylate of an organic or inorganic oxoacid.

O—R may thus suitably be, for example, an alkanoate, preferably a lower alkanoate with 2 to 6 carbon atoms, such as an acetate, glyoxylate, propionate, pyruvate or butyrate, preferably acetate, propionate or butyrate preferably attached in the 2-position to the chitosan oxy group.

The alkanoate moiety may be branched or unbranched, and hence suitable butyrates may be n-butyrate or iso-butyrate. The alkanoate group that is most preferred is acetate. The corresponding group of formula M-O—R— may also be referred to as a salified carboxymethyl group.

O—R may also suitably be, for example an alkanesulphonate, preferably a lower alkanesulphonate with 2 to 6 carbon atoms, such as a methanesulphonate, ethanesulphonate, or propanesulphonate, preferably attached in the 1-position to the chitosan oxy group, and more preferably methanesulphonate. The alkane moiety may be branched or unbranched, and hence suitable propane sulphonates may be propane-1- or 2-sulphonate, and butanesulphonates may be butane-1-sulphonate, 2,2-dimethylethane-1-sulphonate or 1,2-dimethylethane-1-sulphonate. The alkane sulphonate substituent group that is most preferred is methane sulphonate. The corresponding group of formula M-O—R— may also be referred to as a salified sulphonatomethyl group.

O—R may also suitably be an arenecarboxylate, such as a benzoate or toluate, substituted by a nucleophilic leaving group T.

O—R may also suitably be an arenesulphonate, such as benzenesulphonate or toluenesulphonate, substituted by a nucleophilic leaving group T.

Suitable and preferred nucleophilic leaving groups T, when O—R is an arenecarboxylate or an arenesulphonate, and suitable and preferred substitution positions for T will be well-known to the skilled person.

Process steps (a) to (c) in the present invention are a method for the preparation of a derivatised chitosan precursor of the further derivatised chitosan polymer of the present invention, and these a specific form of these steps is described further in our copending application GB 1100426.4 to process steps (a) to (c).

As noted in that application, it s preferred that the process steps a) to c) are carried out at a temperature below 50° C. As regards the reaction temperature, for the process overall it may suitably be 25 to 45° C. For example, about 30 to 40° C. has been found to be suitable to give a useful degree of substitution in an economic time, without deleterious side reactions, and with a product of consequently improved physical properties.

The reaction time for the process overall to give a useful degree of substitution in an economic time may suitably be 0.5 to 8 hours, for example 1 to 5 hours, such as 1 to 3 hours.

As noted above, the derivatised chitosan which is the product of reaction steps (a) to (c) above is one in which one or both of the hydroxyl groups on the D-glucosamine units of the chitosan are converted at least in part to salified O—R—O— groups (etherification), where O—R is a hydrocarbyl oxoacid anion.

In process steps (a) and (b), especially when carried out at a temperature below 50° C., the base is preferably an alkali metal hydroxide, in which case the metal cation is preferably the same as M in the compound of formula (III), preferably an alkali metal hydroxide, such as sodium hydroxide, and these hydroxyl functions are preferably converted to M-O—R—O— groups; and the other hydroxyl groups on the D-glucosamine units of the chitosan are generally converted to hydroxyl group derivatised by the base (alkalisation), preferably M-O— groups.

Either reaction may take place at either hydroxyl position in the chitosan macromolecule, in any distribution up to the maximum degree of substitution that is possible.

Process step d) or a variant thereof may also be carried out independently of process steps a) to c), for example on a derivatised chitosan formed by other methods or of a different composition.

Thus, according to the second aspect of the present invention, there is also provided a second process for producing a further derivatised chitosan as defined, which comprises reacting
a) a derivatised chitosan which is substituted by salified hydroxy-(organic or inorganic acyl)-hydrocarbyloxy groups in place of at least a proportion of the chitosan hydroxyl functions with
b) an alkylene oxide of the formula (IV):

R¹(R²)V(R³)R⁴ (IV)
wherein
V is an ethylene oxide moiety, and
each of R¹, R², R³ and R⁴, is H or an optionally substituted hydrocarbyl group, and R¹ and R² are at the 2-positions, and R³ and R⁴ are at the 3-positions, on V.

As noted hereinafter, the further derivatised chitosan of the invention is often in the form of fibre tow. It is therefore preferred that the derivatised chitosan starting material of this invention is in a form corresponding to that of the desired further derivatised chitosan, in particular as fibres.

It is particularly preferred that the reaction is carried out in the presence of water, as a liquid, optionally absorbed by the derivatised chitosan fibres, and/or as vapour, introduced before or during the reaction. The presence and the quantity of moisture available at that point is a significant factor in the reaction.

In an embodiment which corresponds to step d) of the first process of the invention the reaction comprises reacting
a) a derivatised chitosan which
   i) is substituted in place of at least part of the chitosan hydroxyl groups by salified O—R—O— groups as hereinbefore defined); and
   ii) is substituted in place of at least part of the other chitosan hydroxyl groups hydroxyl groups derivatised by a base (preferably M-O— groups as hereinbefore defined)
      with
b) an alkylene oxide of the formula (IV):

R¹(R²)V(R³)R⁴ (IV)
wherein
V is an ethylene oxide moiety, and
each of R¹, R², R³ and R⁴, is H or an optionally substituted hydrocarbyl group, and R¹ and R² are at the 2-positions, and R³ and R⁴ are at the 3-positions, on V.

As noted hereinafter, the further derivatised chitosan of the invention is often in the form of fibre tow. It is therefore preferred that the derivatised chitosan starting material of this invention is in a form corresponding to that of the desired further derivatised chitosan, in particular as fibres.

It is particularly preferred that the reaction is carried out in the presence of water, as a liquid, optionally absorbed by the derivatised chitosan fibres, and/or as vapour.

Conditions for the reaction with the alkylene oxide of the formula (IV) are described further herein in respect of this reaction, whether carried out as a process step (d) of the first process of the second aspect of the present invention, or as an independent reaction.

The further derivatised chitosan fibre product from the second process of the second aspect of the invention is believed to be one in which on the D-glucosamine units of the derivatised chitosan
a) in place of at least part of the chitosan hydroxyl groups the chitosan is substituted by basified hydroxyethyl ester groups of formula (I)

HO—(R¹)(R²)C—C(R³)(R⁴)—O*—R—O— (I); and.
c) the chitosan amine groups are substituted at least in part by optionally basified groups of formula (IIa):

HO—(R¹)(R²)C—C(R³)(R⁴)— (IIa)

The basifying moiety is preferably a Group IA metal cation M as defined, in particular a sodium cation.

It will be appreciated that if such a further derivatised chitosan is contacted with an aqueous medium, exchange of basifying groups between basified and non-basified HO— groups may occur, so that for example they may transfer from any moiety of formula (I) to that of formula (IIa), and that the definitions of the groups of formulae (I) and (IIa) should be interpreted accordingly.

In the embodiment which corresponds to step d) of the first process of this second aspect, whether operated independently or as step d) of the process the chitosan product (as defined hereinbefore) is believed to be one in which on the D-glucosamine units of the derivatised chitosan
a) in place of at least part of the chitosan hydroxyl groups the chitosan is substituted by basified hydroxyethyl ester groups of formula (I)

HO—(R¹)(R²)C—C(R³)(R⁴)—O*—R—O— (I); and
b) in place of at least part of the other chitosan hydroxyl groups the chitosan is substituted by basified groups of formula (II):

HO—(R¹)(R²)C—C(R³)(R⁴)—O— (II); and.
c) the amine groups are substituted at least in part by optionally basified groups of formula (IIa):

HO—(R¹)(R²)C—C(R³)(R⁴)— (IIa)

The basifying moiety is preferably a Group IA metal cation M as defined, in particular a sodium cation.

It will be appreciated that if such a further derivatised chitosan is contacted with an aqueous medium, exchange of basifying groups between basified and non-basified HO— groups may occur, so that for example they may transfer from any moiety of formula (I) or (II) to that of formula (IIa), and that the definitions of the groups of formulae (I), (II) and (IIa) should be interpreted accordingly.

It is believed that, whilst the alkylene oxide of the formula (IV) as defined hereinbefore in respect of the process of the first aspect of the present invention reacts with both the salified O—R—O— groups, preferably alkali metal salt groups (esterification), the amine groups, and the basified hydroxyl groups, preferably alkali metal oxide, M-O— groups (if present), the former two groups may be more reactive than the last. It is also believed that both the first two reactions are facilitated by the presence of the base (e.g. alkali metal) cations at the oxo acid and hydroxyl group and the presence of moisture.

Such conversion may take place at any salified O—R—O— group or M-O— group (if present) position in the derivatised chitosan macromolecule, in any distribution up to the maximum degree of conversion that is possible.

The reaction of the amine groups with the alkylene oxide of formula (IV) is facilitated by the presence of moisture.

In the further derivatised chitosan produced by the reaction, whether as step (d) of the first process, or the independent second process, according to the second aspect of the present invention, conversion of any salified O—R—O— group or M-O— group (if present) or amine group in the derivatised chitosan macromolecule may take place up to a theoretical 100% the maximum degree of conversion.

The specific average degree of substitution by the respective converted groups will be determined by the specific average degree of substitution by the respective unconverted groups in the derivatised chitosan which is the product of reaction steps (a) to (c) above, or in the derivatised chitosan which is the starting material of the independent second process of the invention.

The further derivatised chitosan should be substantially water-insoluble, in particular for use in absorbent medical devices, such as wound dressings. In general, if the starting material derivatised chitosan is substantially water-insoluble, the further derivatised chitosan will also be so. The average degree of substitution by all substituent groups in the starting material derivatised chitosan is suitably less than 0.8, preferably less than 0.7, more preferably less than 0.6 for the derivatised chitosan to be substantially water-insoluble. The average degree of substitution in the further derivatised chitosan polymer of the present invention may more suitably be from about 0.1 to about 0.4, for example from about 0.15 to about 0.35, such as from about 0.2 to 0.3. The average molecular weight of the chitosan is often between 3800 to 20,000 daltons

It will also be appreciated that the composition of the starting material derivatised chitosan may vary with the properties of its starting material chitosan, including its source, the degree of deacetylation of the chitin starting material that is reached in its production, and its molecular weight. The degree of deacetylation is often from 60 to 100%, and the average molecular weight is often between 3800 to 20,000 daltons.

Step (d) of the first process or the independent second process is an epoxidation process, which is often carried out as a single step in which the reactants are added at the same time in one reaction vessel.

It will be clear to those skilled in the art that the form of the derivatised chitosan starting material of steps (a) to (c) of the process may have a significant effect on the form of the further derivatised chitosan product.

It will be appreciated that many forms or modifications of the starting material derivatised chitosan may of course be used to produce suitable products for use in absorbent, in particular wound care medical, devices, and these fall within the scope of the present invention.

An unmodified or modified related material, such as a chitin, may also be used as a starting material in the present process. All of these materials may be modified in the present process to produce absorbent products, for example for use in wound care medical devices. For example, a natural chitosan derived from shrimp or snow crab and/or a natural chitin may be utilised.

The further derivatised chitosan is often in the form of fibre tow. The fibres may be used in a wide range of lengths, for example a few mm, such as 2 mm to 5 mm, to several tens of mm, for example 100 mm or more. It will be clear to those skilled in the art that the physical form of the derivatised chitosan and underivatised starting materials will have a significant effect on the physical form of the derivative chitosan product.

As described further hereinafter in respect of the processes of the second, fifth and seventh aspects of the present invention for the further derivatisation of a derivatised chitosan, and in the following non-limiting examples, respectively per se, in a non-woven fabric, and in an absorbent device, moisture is preferably present in the starting material of, or introduced during, the reaction. The presence and the quantity of moisture available at that point is a significant factor in the reaction.

Epoxidation is often carried out with a prior preconditioning step, in which the derivatised chitosan, whether it is the product of reaction steps (a) to (c) above or otherwise derived, is first equilibrated in air. This may suitably be carried out at a temperature up to 65° C., which may suitably be 30 to 60° C., for example 45 to 55° C. and a relative humidity of 25 to 95%, such as 35 to 85%, for example 45 to 75%. The time for this equilibration prior to the epoxidation process may suitably be 1 to 18 hr, such as 5 to 16 hr. 9 to 14 hr is found to be most suitable to give a more useful substrate for processing in an economic time.

After this preconditioning step, if the derivatised chitosan has not been equilibrated in the reaction vessel in which epoxidation is to be carried out, it is transferred to the vessel. Before epoxidation is carried out, the vessel is purged of air to introduce an inert atmosphere, by for example evacuating it and introducing nitrogen into the vessel.

The derivatised chitosan is then subjected to treatment with an alkylene oxide of the formula (IV):

R¹(R²)V(R³)R⁴ (IV)

wherein
V is an ethylene oxide moiety, and
each of R¹, R², R³ and R⁴, is H or an optionally substituted hydrocarbyl group, and
R¹ and R² are at the 2-positions, and R³ and R⁴ are at the 3-positions, on V.

The reaction is suitably effected in a fluid—solid reaction step, preferably in a gas—solid reaction step, and optionally in a mixture of water vapour and vapour of the alkylene oxide of the formula (IV).

In the alkylene oxide of the formula (IV):

Each of R¹, R², R³ and R⁴ is H or a hydrocarbyl group, preferably H.

Each of R¹, R², R³ and R⁴ independently may suitably be an alkyl group, preferably an optionally substituted lower alkyl group with 1 to 6 carbon atoms, such as a methyl, ethyl or propyl, preferably methyl.

The alkyl moiety may be branched or unbranched, and hence suitable propyl may be n-propyl or iso-propyl.

Each of R¹, R², R³ and R⁴ independently also suitably be, for example an acyl group, preferably a lower acyl group with 1 to 6 carbon atoms, such as a formyl or acetyl group. Preferably all of R¹, R², R³ and R⁴ together then have 1 to 6 carbon atoms, preferably 1.

Each of R¹, R², R³ and R⁴ independently may also suitably be an aryl group, such as phenyl or tolyl, preferably phenyl. Preferably only one of R¹, R², R³ and R⁴ is phenyl.

Most preferably each of R¹, R², R³ and R⁴ is H.

Generally, the higher the concentration (partial pressure) of the alkylene oxide, the faster the rate of reaction and the degree of etherification.

However, as noted above, epoxidation is often carried out with an alkylene oxide of the formula (IV), in which each of R¹, R², R³ and R⁴ is H or a hydrocarbyl group, preferably H, and the reaction is preferably effected in a gas—solid reaction step, and optionally in a mixture of water vapour and vapour of the alkylene oxide of the formula (IV).

The concentration of the oxide should be balanced with its nature and boiling point. For example, ethylene oxide or butylene-1,2-oxide in the form of their respective vapours may be utilised. The former has a boiling point of about 11° C., the latter of about 63° C.

Thus, for example, the epoxidation is preferably carried out under a reduced pressure, and a partial pressure of the alkylene oxide in the reaction vessel of, for example, 200 to 500 mbar, such as 250 to 550 mbar, for example 300 to 400 mbar.

It is carried out a temperature up to 65° C., which may suitably be 30 to 60° C., for example 45 to 55° C. has been found to be generally suitable to give a useful degree of conversion in an economic time.

The reaction time for this step (d) of the process may suitably be 1 to 8 hours, such as 1.5 to 4.5 hr. 2 to 2.5 hr is found to be most suitable to give a useful degree of conversion ion in an economic time.

Fresh charges of the oxide can be introduced at any time throughout the reaction. The rate and degree of conversion can be controlled in particular by control of the reaction time and partial pressure.

As noted above, the reaction is preferably effected in a gas—solid reaction step, and optionally in a mixture of water vapour and vapour of the alkylene oxide of the formula (IV). The water vapour may be introduced by for example evacuating the nitrogen from the vessel and introducing steam into it before the derivatised chitosan is subjected to etherification with the alkylene oxide of the formula (IV). The steam may also be used to heat the vessel and the derivatised chitosan to the desired reaction temperature.

A partial pressure of water vapour in the reaction vessel of, for example, 15 to 60 mbar, such as 20 to 55 mbar, for example 25 to 50 mbar, at a temperature and over a reaction time as referred to above. Fresh charges of water vapour can be introduced at any time throughout the reaction.

The reaction with the compound of formula (IV) may be catalysed by the presence of water vapour, in particular where the derivatised chitosan contains hydroxyl groups etherified by an alkali metal hydroxide, in which case the metal cation is released by the water, to catalyse the reaction. It is possible that the rate and degree of conversion can be controlled in particular by controlling the partial pressure of water vapour.

After this reaction step, the reaction vessel should be evacuated to remove residual fluids from the further derivatised chitosan product, and the chitosan should be gas washed by employing gas washing stages known in the art in the reaction vessel in which etherification has been carried out.

At least 2 washes in an inert atmosphere, by for example evacuating and introducing nitrogen into the vessel, and at least 8 air washes by the same method are preferred. For example, 20 to 40 min per wash may be suitable. Finally the derivatised chitosan should be transferred out of the vessel.

Drying should not be necessary, but if it is, simple drying can be carried out by methods known in the art such as forced air drying, radiant heat drying etc.

The simplicity of the chemistry and the availability of the reactants enable the cost of manufacture of medical and other devices (as hereinbefore defined) to be kept advantageously low.

In a third aspect, the present invention provides a further derivatised chitosan produced by step (d) of the first process, or by the second process, according to the second aspect of the present invention. The product is preferably in the form of fibres, and the specific form of the fibres will often be determined by the specific from of the starting material of the first or second process of the second aspect of the present invention.

The further derivatised chitosan fibres of the first aspect of the invention are often processed into and comprised in a non-woven fabric, often for use in an absorbent device.

The dry strength of the further derivatised fibres is sufficient to enable processing into woven or preferably non-woven structures, and, to be useful as an absorbent material in an absorbent device, in particular a wound dressing, the wet strength of the material is sufficient to allow removal from the site in one piece.

The pre-absorption further derivatised chitosan fibres according to the present invention may have a monofilament linear density of 0.1 to 30, preferably about 0.5 to 20, and more preferably 0.9 to 8, for example 1 3 to 5 decitex, and a strength of 0.8 to 2.2, such as 1 to 2, for example 1.2 to 1.8 cN/dtex.

Whilst they swell in contact with water to become an elastic gel material, and exhibit good absorption and retention of fluid, they maintain their integrity sufficiently, for example in wound dressings, to be removed from the wound site in one piece, without irrigation, and with minimum pain and shedding. Absorption of fluid is virtually instantaneous since ionic exchange is not required for the fibres to become gellable.

However, the initial aqueous absorbency of the further derivatised chitosan fibre is not so high that it causes difficulty in fibre opening and web formation during subsequent processing of the further derivatised chitosan fibre to a non-woven fabric by a non-woven technique. Moreover, the water-insoluble further derivatised chitosans of the present invention are also advantageous compared to the prior art carboxymethyl chitosans because the absorptive capacity is not adversely affected by processing into non-woven components for absorbent devices, for example wound dressings.

They are also affected to a lesser extent in use by changes in pH. Wound dressings containing these materials continue to absorb to a good level at low pH.

The air-dried further derivatised chitosan fibre (including any such additive materials) may thus be converted into a non-woven fabric by fibre opening, web formation and needling, which may suitably be of 30 to 200 g/m² or more.

The further derivatised chitosans of the present invention may be processed according to known methods into a wide variety of forms, depending on their intended use, for example as a non-woven component of an absorbent device. The manner in which the derivative chitosan is processed has a significant effect on the properties of the final product, particularly the strength, gelling time, and absorbency.

A fourth aspect of the present invention thus provides an absorbent non-woven fabric comprising further derivatised chitosan fibres of the first aspect of the invention.

As noted above, the overall absorptive capacity of the fabric is sensitive to the sizes and interconnectivity of the inter-fibre volumes within it, and hence to the parameters of the process for its manufacture, and will often differ from that of the component fibres. The therapeutically active, absorbent non-woven fabrics of the present invention comprising further derivatised chitosan fibres meet the criteria for adequate and good absorbency of fluids in absorbent devices, and in particular in advanced wound dressings. The non-woven fabrics comprising further derivatised chitosan fibres may suitably have an absorbency of 8 to 30 grams per gram (g/g) of saline solution, often 12 to 27 g/g, and more often 16 to 23 g/g.

In many embodiments of non-woven fabrics comprising the water-insoluble further derivatised chitosan according to the invention, for example for absorbent devices, it is the only absorbent material present. Such embodiments do not contain other absorbent materials such as hydrogels, anion-exchange resins, etc. or relatively non-absorbent, for example strengthening polymeric, materials In other embodiments of non-woven fabrics comprising the water-insoluble further derivatised chitosan according to the invention, for example for absorbent devices, however, other materials are present. Such embodiments may contain other absorbent materials such as hydrogels, anion-exchange resins, etc. or relatively non-absorbent, for example strengthening polymeric materials, often in the form of fibres which are intermingled with the further derivatised chitosan according to the invention.

Thus, another embodiment of the present invention is directed to a non-woven fabric comprising a further derivatised chitosan of the present invention which is reinforced with a reinforcing fibre blended with the water-insoluble further derivatised chitosan.

Examples of other materials that may be present include other absorbent materials, such as hydrogels, for example an alginate, or anion-exchange resins, or a mixture thereof; or relatively non-absorbent, for example polymeric strengthening, materials, such as unmodified chitosan or thermoplastic bicomponent fibres, most preferably having a polyolefin component, for example comprising a polyolefin-containing polymeric material of which the largest part (by weight) consists of homo- or copolymers of monoolefins such as ethylene, propylene, 1-butene, 4-methyl-l-pentene, etc.

Preferably, such materials are added after drying. For example, un-modified chitosan fibre may be added, in a weight ratio of chitosan fibre to the derivatised chitosan fibre of 1:9 to 9:1, for example of 1:6 to 6:1 or 1:3 to 3:1. It will be clear to those skilled in the art that the addition of unmodified chitosan to the derivatised chitosan material may have a significant improving effect on the strength of the material in a non-woven wound dressing.

Preferably, such materials are added after drying. For example, un-modified chitosan fibre may be added, in a weight ratio of chitosan fibre to the further derivatised chitosan fibre of 1:9 to 9:1, for example of 1:3 to 3:1. The air-dried further derivatised chitosan fibre including such additive materials may then be converted into a non-woven fabric as for derivatised chitosan fibre without such additive materials by fibre opening, web formation and needling, which may suitably be of 30 to 200 g/m² or more.

It will be clear to those skilled in the art that the addition of unmodified chitosan to the further derivatised chitosan material may have a significant improving effect on the strength of the material in a non-woven wound dressing.

The absorbent non-woven fabric of the third aspect of the present invention may also be provided by an alternative process for its manufacture.

This alternative process forms a fifth aspect of the present invention, and is a variant of the first or second process according to the second aspect of the present invention for producing a further derivatised chitosan.

In this alternative process, the derivatised chitosan intermediate of steps (a) to (c) of the first process or starting material of the second process is processed according to known methods into a non-woven fabric, before being reacted with an alkylene oxide of the formula (IV):

R¹(R²)V(R³)R⁴ (IV)

wherein
V is an ethylene oxide moiety, and
each of R¹, R², R³ and R⁴, is H or an optionally substituted hydrocarbyl group, and
R¹ and R² are at the 2-positions, and R³ and R⁴ are at the 3-positions, on V.

As noted above, the term ‘derivatised chitosan’ as used herein refers to the product of reaction steps (a) to (c) above in the first process, or the starting material in the second process, according to the second aspect of the present invention. In the present process variant, the derivatised chitosan is comprised in a non-woven fabric.

As also noted above, the term ‘further derivatised chitosan’ as used herein refers to the product of reaction step (d) in the first process, or of the second process, according to the first aspect of the present invention.

Conditions for reaction with an alkylene oxide of the formula (III) are similar to those described further hereinbefore in respect of the processes of the second aspect of the present invention for the further derivatisation of a derivatised chitosan.

As with non-woven fabrics comprising the further derivatised chitosan of the first aspect of the invention, in a non-woven fabric comprising the water-insoluble derivatised chitosan which is the process starting material, it may be the only absorbent material present. In other non-woven fabrics comprising the water-insoluble derivatised chitosan which are the process starting materials, other materials may be present.

Such embodiments may contain other absorbent materials such as hydrogels, anion-exchange resins, etc. or relatively non-absorbent, for example strengthening polymeric materials, often in the form of fibres which are intermingled with the further derivatised chitosan according to the invention.

The manner in which the derivatised chitosan is first processed after steps (a) to (c) may have a significant effect on the properties of the final non-woven product of step (d), particularly the strength, gelling time, and absorbency.

As noted above, the overall absorptive capacity of such a fabric is sensitive to the sizes and interconnectivity of the inter-fibre volumes within it, and hence to the parameters of the process for its manufacture, and will often differ from that of the component fibres. The starting material non-woven fabric for step (d) may be prepared from air-dried derivatised chitosan fibre (including any additive materials as above) by fibre opening, web formation and needling, which may suitably give rise to a non-woven of 30 to 200 g/m² or more.

The therapeutically active, absorbent non-woven fabrics produced by the process of the fifth aspect of the present invention comprising further derivatised chitosan fibres also meet the criteria for adequate and good absorbency of fluids in absorbent devices, and in particular in advanced wound dressings. The non-woven fabrics comprising further derivatised chitosan fibres may suitably have an absorbency of 8 to 30 grams per gram (g/g) of saline solution, often 12 to 27 g/g, and more often 16 to 23 g/g.

Their absorbent properties, biodegradability, and the fact that chitosan is a renewable material, mean that the further derivatised chitosan absorbent materials of the present invention may be used in a wide range of absorbent devices.

It will be clear to those skilled in the art that the area density of the product of the processing may have a significant effect on the absorbency of the processed further derivatised chitosan material.

The non-woven fabric (prepared by either of the above routes) may then be converted to an anti-microbial absorptive component of a non-woven absorptive device by cutting, packaging and sterilising.

In a sixth aspect of the present invention there is provided an absorbent device (as defined hereinbefore) comprising further derivatised chitosan fibres of the second aspect of the invention.

As noted above, the overall absorptive capacity of the fabric in the device is sensitive to the sizes and interconnectivity of the inter-fibre volumes within it, and hence to the parameters of the process for its manufacture, and will often differ from that of the component fibres. The therapeutically active, absorbent non-woven fabrics of the present invention comprising further derivatised chitosan fibres meet the criteria for adequate and good absorbency of fluids in absorbent devices, and in particular in advanced wound dressings. The non-woven fabrics comprising further derivatised chitosan fibres have an absorbency of 8 to 30 grams per gram (g/g) of saline solution, often 12 to 27 g/g, and more often 16 to 23 g/g.

In absorbent devices comprising non-woven fabrics comprising the water-insoluble derivatised chitosan which is the process starting material, it may be the only absorbent material present. In other non-woven fabrics comprising the water-insoluble derivatised chitosan which are the process starting materials, other materials may be present. Such embodiments may contain other absorbent materials such as hydrogels, anion-exchange resins, etc. or relatively non-absorbent, for example strengthening polymeric materials, often in the form of fibres which are intermingled with the further derivatised chitosan according to the invention.

Their absorbent properties, biodegradability, and the fact that chitosan is a renewable material, mean that the further derivatised chitosan absorbent materials of the present invention may be used in a wide range of absorbent devices. The absorbent materials of the present invention exhibit instant gelling in aqueous media, good absorbency and, crucially, good retention of absorbency in an acidic environment.

When fully hydrated, the absorbent medical device is substantially transparent. This is advantageous in wound care applications since the state of the underlying wound can be determined without removing the dressing.

This renders them ideal for use as an absorbent wound care product, such as a dressing, or as part of an absorbent wound care product. They are suited for use in wound dressings for acute wounds, including surgical wounds, and chronic and burn wounds.

They are particularly useful for wounds with moderate to high levels of exudates, and for flat or cavity wounds of this type. Typical examples include burn wounds, and chronic wounds, such as pressure sores and leg ulcers. The dressing, when covering a wound, is able to prevent water in body fluids from being lost, providing a favourable moist environment for wound healing and maintaining a fluid-free, maceration-free, germ-free wound surface.

They may also be used in ostomy devices, and surgical and dental sponges.

Thus, one embodiment of this sixth aspect of the present invention provides an absorbent medical device comprising the further derivatised chitosan fibres of the first aspect of the invention.

Their absorbent properties, biodegradability, and the fact that chitosan is a renewable material, mean that the further derivatised chitosan absorbent materials of the present invention may be used in a wide range of absorbent devices.

Preferred further derivatised chitosan products for use in wound care medical devices are carded, needle-bonded non-wovens.

The further derivatised chitosan which is comprised in a non-woven fabric, in turn comprised in an absorbent device or absorbent component thereof, as described further hereinbefore, may suitable be present as about 10 to 13%) of the absorbent medical device. They preferably have a pre-absorption monofilament linear density of 0.1 to 30, preferably about 0.5 to 20, and more preferably 0.9 to 8, for example 1 3 to 5 decitex, and a strength of 0.8 to 2.2, such as 1 to 2, for example 1.2 to 1.8 cN/dtex.

Whilst they swell in contact with water to become an elastic gel material, and exhibit good absorption and retention of fluid, they maintaining their integrity sufficiently, for example in wound dressings, to be removed from the wound site in one piece, without irrigation, and with minimum pain and shedding. Absorption of fluid is virtually instantaneous since ionic exchange is not required for the fibres to become gellable.

The use of the absorbent materials of the present invention is not limited to medical products, however, and they are useful for other applications.

Their absorbent properties, biodegradability, and the fact that chitosan is a renewable material, mean that absorbent devices comprising the further derivatised chitosans of the invention are also particularly desirable for use in absorbent pads for the personal care sector, particularly for disposable sanitary devices such as nappies (diapers), disposable nappies and training pants, feminine care products, for example, tampons, sanitary towels, or napkins and pant liners, and incontinence products.

Thus, another embodiment of this sixth aspect of the present invention provides an absorbent personal care device comprising the further derivatised chitosan fibres of the second aspect of the invention.

The absorbent device of the sixth aspect of the present invention may also be provided by an alternative process for its manufacture, which forms a seventh aspect of the present invention, and is a variant of the first or second process according to the second aspect of the present invention for producing a further derivatised chitosan.

In this variant, the derivatised chitosan intermediate of steps (a) to (c) of the first process or starting material of the second process is processed according to known methods into a non-woven fabric, which is in turn processed according to known methods into an absorbent device or absorbent component thereof, before being reacted with an alkylene oxide of the formula (IV):

R¹(R²)V(R³)R⁴ (IV)

wherein
V is an ethylene oxide moiety, and
each of R¹, R², R³ and R⁴, is H or an optionally substituted hydrocarbyl group, and
R¹ and R² are at the 2-positions, and R³ and R⁴ are at the 3-positions, on V.

As noted above, the term ‘derivatised chitosan’ as used herein refers to the product of reaction steps (a) to (c) above in the first process, or the starting material in the second process, according to the second aspect of the present invention. In the present process variant, the derivatised chitosan is comprised in a non-woven fabric, in turn comprised in an absorbent device or absorbent component thereof.

As also noted above, the term ‘further derivatised chitosan’ as used herein refers to the product of reaction step (d) in the first process, or of the second process, according to the second aspect of the present invention.

Conditions for reaction with an alkylene oxide of the formula (III) are similar to those described further hereinbefore in respect of the process of the first aspect of the present invention for the further derivatisation of a derivatised chitosan.

In an eighth aspect the invention provides a method of use of an absorbent device according to the present invention. In one embodiment, it provides a method of treatment of the human or animal body using an absorbent device according to the present invention, in particular in wound care.

The invention will now be illustrated by the following non-limiting examples.

### EXAMPLE 1

This example demonstrates steps (a) to (c) of the process for making super-absorbent gelling, water insoluble further derivatised chitosan fibres by carboxymethylation of the fibres.

A solution containing 10.625 kg sodium hydroxide (40% solution), 4.5 kg sodium chloroacetate (44% solution) and 100 kg ethanol (60% solution) was prepared, the remainder being deionised water. The solution was heated to 30° C. 5.0 kg chitosan fibres were added and the solution reacted for 45 minutes. The temperature of the solution was then increased to 40° C., and the fibres were reacted for a further 120 minutes. The modified fibres underwent 5 spin dry and ethanol wash cycles and a final spin dry. The fibres were treated with a spin finish comprised 1.523% Tween 20 and 98.478% ethanol (95%).

The fibres underwent another spin dry after fibre finish application and were left to air dry and condition.

### EXAMPLE 2

This example demonstrates step (d) of the process for making super-absorbent gelling, water insoluble further derivatised chitosan fibres by etherification of the fibres with ethylene oxide (oxirane).

In a pressure vessel, the product of Example 1 was equilibrated in air at a temperature of 45 to 55° C. and a relative humidity of 45 to 75% for 12 hr. After this preconditioning step, the vessel was purged of air to introduce an inert atmosphere, by evacuating it and introducing nitrogen into the vessel. The vessel was then re-evacuated and a partial pressure of steam (42 mbar) to heat the reaction vessel to 45 to 55° C. was introduced, followed by ethylene oxide at a partial pressure of 300 to 400 mbar, at a temperature maintained at 45 to 55° C., and held there for a reaction time of 2 hr.

The product chitosan was then gas washed by employing gas washing stages known in the art in the reaction vessel, that is, 2 washes by evacuating and introducing nitrogen into the vessel, and 8 air washes by the same method.

### EXAMPLE 3

The gram per gram absorbency of a non-woven fabrics made from the product of Example 2 was determined as follows:

Three fabric specimens were cut to 5 cm×5 cm weighed. Each specimen was then placed in a Petri dish and covered with an excess of Solution A.

Solution A is a specific solution of sodium chloride and calcium chloride QCP090 as used in British/European Standard BS EN 13726-1:2002 Test methods for primary wound dressings Part 1 Aspect of absorbency.) Each specimen was left in the Solution A for 180 seconds then removed from the Solution A with forceps, and allowed to drain for 15 seconds. Each specimen was then weighed.

The gram per gram absorbency of the derivatised chitosan fibres before and after conversion in Example 2 to super-absorbent gelling, water insoluble further derivatised chitosan fibres was found to be respectively 18 g/g and 24 g/g.

1. A further derivatised chitosan wherein in place of at least part of the chitosan hydroxyl groups the chitosan is substituted by basified hydroxyethyl ester groups of formula (I)
HO—(R¹)(R²)C—C(R³)(R⁴)—O*—R—O— (I)
wherein each of R¹, R², R³ and R⁴ is H or an optionally substituted hydrocarbyl group, and
R is an organic or Inorganic acyl hydrocarbyl residue, orientated such that the basified HO—(R¹)(R²)C—C(R³)(R⁴)— moiety forms an ester linkage with the oxygen atom marked *.

2. A chitosan according to claim 1 wherein
a) in place of at least part of the chitosan hydroxyl groups the chitosan is substituted by basified hydroxyethyl ester groups of formula (I)
HO—(R¹)(R²)C—C(R³)(R⁴)—O*—R—O— (I); and
b) the chitosan amine groups are substituted at least in part by optionally basified groups of formula (IIa):
HO—(R¹)(R²)C—C(R³)(R⁴)— (IIa)
wherein R¹, R², Wand R⁴ are as defined in claim 1.

3. A chitosan according to claim 1 wherein
a) in place of at least part of the chitosan hydroxyl groups the chitosan is substituted by basified hydroxyethyl ester groups of formula (I)
HO—(R¹)(R²)C—C(R³)(R⁴)—O*—R—O— (I); and
b) in place of at least part of the other chitosan hydroxyl groups the chitosan is substituted by basified groups of formula (II):
HO—(R¹)(R²)C—C(R³)(R⁴)—O— (II); and.
c) the amine groups are substituted at least in part by optionally basified groups of formula (IIa):
HO—(R¹)(R²)C—C(R³)(R⁴)— (IIa)
wherein R¹, R², R³ and R⁴ are as defined in claim 1.

4. A chitosan according to claim 1 wherein the O*—R moiety is an acetate residue.

5. A chitosan according to claim 1 wherein the O*—R moiety is a methanesulphonate residue.

6. A process for producing a chitosan according to claim 1, which comprises reacting
a) a derivatised chitosan which is substituted by salified hydroxy-(organic or inorganic acyl)-hydrocarbyloxy groups in place of at least a proportion of the chitosan hydroxyl functions.
with
b) an alkylene oxide of the formula (III):
R¹(R²)V(R³)R⁴ (IV)
wherein
V is an ethylene oxide moiety, and
each of R¹, R², R³ and R⁴, is H or an optionally substituted hydrocarbyl group, and R¹ and R² are at the 2-positions, and R³ and R⁴ are at the 3-positions on V.

7. A process according to claim 6 wherein the derivatised chitosan is produced by a process which comprises contacting chitosan fibre
(a) with a base in an aqueous or non-aqueous medium, and
(b) with a solution in an aqueous or non-aqueous medium of a salt of the formula (III):
M-O—R-T (III)
wherein
M is a Group IA metal cation;
O—R is a hydrocarbyl oxoacid anion residue; and
T is a nucleophilic leaving group; and
(c) isolating and washing the product of steps (a) and (b).

8. A process according to claim 7 in which steps (a) to (c) are all carried out at a temperature below 50° C.

9. A process according to claim 6, wherein the moiety —O—R is an acetate residue.

10. A process according to claim 8, wherein the moiety —O—R is a methanesulphonate residue.

11. A process according to claim 6, wherein R¹(R²)V(R³) R⁴ is ethylene oxide.

12. A process according to claim 6, which is carried out with a mixture of water vapour and vapour of the alkylene oxide of the formula (IV).

13. A process according to claim 6, which is carried out at a temperature of up to 65° C.

14. A further derivatised chitosan which is the product of the process according to claim 6 or 7.

15. An absorbent non-woven fabric comprising a further derivatised chitosan according to claim 1.

16. A process for preparing a non-woven fabric according to claim 15, which comprises reacting a non-woven fabric comprising a derivatised chitosan, substituted by salified hydroxy-(organic or inorganic acyl)-hydrocarbyloxy groups in place of at least a proportion of the chitosan hydroxyl functions, with an alkylene oxide of the formula (IV):
R¹(R²)V(R³)R⁴ (IV)
wherein
V is an ethylene oxide moiety, and
each of R¹, R², R³ and R⁴, is H or an optionally substituted hydrocarbyl group, and R¹ and R² are at the 2-positions, and R³ and R⁴ are at the 3-positions on V.

17. An absorbent device comprising a further derivatised chitosan according to claim 1.

18. A process for preparing an absorbent device according to claim 17, which comprises reacting an absorbent device comprising a derivatised chitosan substituted by salified hydroxy-(organic or inorganic acyl)-hydrocarbyloxy groups in place of at least a proportion of the chitosan hydroxyl functions, with an alkylene oxide of the formula (IV):
R¹(R²)V(R³)R⁴ (IV)
wherein
V is an ethylene oxide moiety, and
each of R¹, R², R³ and R⁴, is H or an optionally substituted hydrocarbyl group, and R¹ and R² are at the 2-positions, and R³ and R⁴ are at the 3-positions on V.

19. The use of an absorbent device according to claim 17 in wound care.
